(19) United States
(12) Patent Application Publication
(10) Pub. No.: US 2020/0408748 A1
(43) Pub. Date: Dec. 31, 2020

(54) Antibody Substance Detection

(71) Applicant: Kevin Brian Delahoussaye, Las Cruces, NM (US)

(72) Inventor: Kevin Brian Delahoussaye, Las Cruces, NM (US)

(21) Appl. No.: 16/458,165

(22) Filed: Jun. 30, 2019

(51) Int. Cl. G01N 33/543

(52) U.S. Cl. CPC G01N 33/543

(57) ABSTRACT

When anti-bodies recognize any foreign substance in the body it creates a copy to replace light variable chains of the Anti-Body; specific to, I say with most certainty, every substance you ingest. The presence of any substance in your body (drugs, proteins, allergens, haptins, ect) creates an humoral response which creates antibodies specific to the substance. A molecule specific to the antibody in question is attached to a surface and the antibody in question binds to that molecule. This novel technique is the purpose behind the patent.

### CROSS REFERENCE TO RELATED APPLICATIONS

Not Applicable Description:

### REGARDING FEDERALLY SPONSORED RESEARCH OR DEVELOPMENT

Not Applicable

### REGARDING THE NAMES OF THE PARTIES TO A JOINT RESEARCH AGREEMENT

Not Applicable

### INCORPORATION BY REFERENCE OF MATERIAL SUBMITTED ON A COMPACT DISC OR AS A TEXT FILE VIA THE THE OFFICE ELECTRONIC FILING SYSTEM (EFS-WEB)

### STATEMENT REGARDING PRIOR DISCLOSURES BY THE INVENTOR OR A JOINT INVENTOR

### BACKGROUND OF INVENTION

The mixing of the principles of organic chemistry and immunology to provide a template regarding the production of antibodies and the detection of them.

### BRIEF SUMMARY OF INVENTION

When anti-bodies recognize any foreign substance in the body it creates a copy to replace light variable chains of the Anti-Body; specific to, I say with most certainty, every substance you ingest. The information for this variable-chain creation is stored in a mathematically racemic structure.

### Amended*

### DETAILED DESCRIPTION OF INVENTION

The presence of any substance in your body (drugs, proteins, allergens, haptins, ect) creates an humoral response which creates antibodies specific to the substance.
if the substance is novel innate immunity follows
if the substance is known your body produces an adaptive response.

Testing of any substance can be determined with this method at exceptionally low concentrations and with maximum certainty.

### Antibody Substance Detection

### Intended Use

A molecule specific to the antibody in question is attached to a surface and the antibody in question binds to that molecule. The antibody is by targeting specific sections of the antigen in question. These sections are distinguishable with the knowledge of organic chemistry.

### Amended*

### CLAIM OR CLAIMS

See Document “Claims”

### ABSTRACT OF DISCLOSURE

See Document “Abstract”

### SEQUENCE LISTING

Not Applicable

Find a pictorial description attached.

1: When anti-bodies recognize any foreign substance in the body it creates a copy to replace light variable chains of the Anti-Body; specific to, I say with most certainty, every substance you ingest. This allows for detection and testing of immune response to a substance. Information can be gathered by the path the immune system follows.

2: A molecule specific to the antibody in question is attached to a surface and the antibody in question binds to that molecule. The antibody is by targeting specific sections of the antigen in question.
